(19) United States
(12) Patent Application Publication
(10) Pub. No.: US 2005/0234052 A1
(43) Pub. Date: Oct. 20, 2005

(54) Treatment for ocular infections

(76) Inventor: J. Greg Slatter, Chester, NJ (US)

Correspondence Address: PHARMACIA & UPJOHN, 301 HENRIETTA ST, 0228-32-LAW, KALAMAZOO, MI, 49007, US

(21) Appl. No.: 11/159,897

(22) Filed: Jun. 23, 2005

Related U.S. Application Data
(63) Continuation of application No. 10/238,449, filed on Sep. 10, 2002.
(60) Provisional application No. 60/322,153, filed on Sep. 14, 2001.
(60) Provisional application No. 60/322,115, filed on Sep. 14, 2001.

(51) Int. Cl. A61K 31/541; A61K 31/5377

(52) U.S. Cl. 514/227.5; 514/235.2

(57) ABSTRACT

The invention provides a method for treating ocular infections such as endophthalmitis.

### CROSS-REFERENCE TO RELATED APPLICATIONS

This application claims the benefit of U.S. Provisional Patent Application No. 60/322,153 filed on Sep. 14, 2001, and No. 60/322,115 filed on Sep. 14, 2001 under 35 U.S.C. § 119(e)(i). The entire disclosures of which are herein incorporated by reference.

### FIELD OF THE INVENTION

The invention relates to a method of treating ocular infections, and more particularly to treating endophthalmitis.

### BACKGROUND

Endophthalmitis involves inflammation of the intraocular cavities, usually caused by an infection. Noninfectious (sterile) endophthalmitis may be a result of retained lens material and toxic agents. Panophthalmitis is inflammation of all coats of the eye including intraocular structures. Although endophthalmitis occurs infrequently, the visual morbidity is high even with appropriate treatment.

There are two types of endophthalmitis, endogenous (ie, metastatic) and exogenous. Endogenous endophthalmitis results from the hematogenous spread of organisms from a distant source of infection (e.g., endocarditis). Exogenous endophthalmitis results from direct inoculation of organisms (e.g., gram-positive coagulase-negative cocci including Staphylococcus epidermidis and Staphylococcus aureus) as a complication of ocular surgery, foreign bodies, or blunt or penetrating ocular trauma.

Exogenous endophthalmitis is commonly encountered after cataract extraction. Cataract extraction is one of the most commonly performed operations in the United States with approximately 1.5 million procedures performed annually. A recent 1994 meta-analysis of the literature showed that the pooled percentage of eyes experiencing endophthalmitis (weighted by sample size and, when pertinent, by quality score of the individual studies but not adjusted for variation in duration of follow-up) was 0.13% which translates to nearly 2,000 cases of endophthalmitis after cataract surgery in the United States.

### SUMMARY

In general, the invention provides a method of treating ocular infections such as endophthalmitis by systemically administering a therapeutically effective amount of a compound of formula I 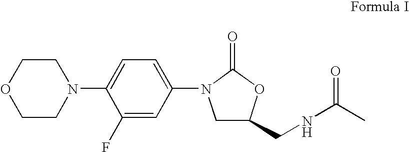
or pharmaceutically acceptable salts thereof. The compound of formula I can be a component of a pharmaceutical composition.

In another aspect, the invention provides a method of treating ocular infections such as endophthalmitis by systemically administering a therapeutically effective amount of a compound of formula II 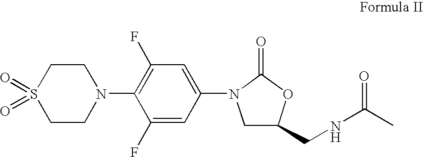
or pharmaceutically acceptable salts thereof. The compound of formula II can be a component of a pharmaceutical composition.

Examples of systemic administration of pharmaceutical compositions containing one or both compounds of formula I and II include oral and intravenous routes. For instance, the pharmaceutical compositions can be administered orally as a tablet or capsule containing a therapeutically effective amount of the compound of formula I or II, or mixtures thereof. In other situations, the pharmaceutical composition can be administered parenterally via an intravenous injection or transdermally, such as by allowing one or both compounds of formula I and II to be absorbed into the blood stream. The pharmaceutical composition can contain from about 0.5% to about 90% weight percent of the compound of formula I or II, or mixture thereof. For instance, the composition can contain about 1 mg to about 1000 mg of the compound of formula I or II, or mixtures thereof. Typically, the pharmaceutical composition includes between about 200 and about 800 mg, e.g., about 600 mg, of the compound of formula I or II, or mixtures thereof. In general, one or both compounds of formula I and II are administered to a mammal, such as a human or animal, in an amount from about 0.1 to about 100 mg/kg of body weight/day.

Advantageously, the compound of formula I or II, or mixtures thereof, when systemically administered, unexpectedly exhibits intraocular penetration, e.g., pass through the blood-aqueous and blood-retinal barriers into the aqueous humor and vitreous. Typically, identifying, a priori, whether or not a specific drug will exhibit intraocular penetration is difficult since penetration through the blood-aqueous and blood-retinal barriers is not entirely predictable. Surprisingly, after systemic administration, the compound of formula I not only penetrates into the aqueous humor and vitreous but also reaches concentrations sufficient to treat bacterial infections. Systemic administration of one or both compounds of formula I and II can lead to aqueous humor and vitreous concentrations that are higher, e.g., about 10 times greater in the aqueous humor and about 100 times greater in the vitreous, relative to topical applications of one or both compounds of formula I and II on the eye.

### DETAILED DESCRIPTION

The compounds, 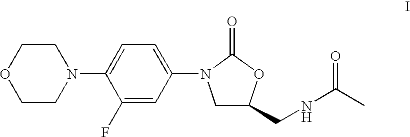
i.e., linezolid, and 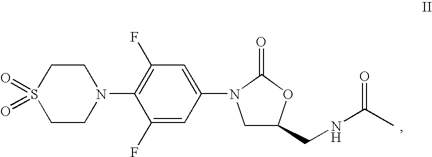
are antimicrobial agents within a new class of antibiotics, the oxazolidinones. Oxazolidinones are effective in the treatment of aerobic Gram-positive bacterial infections by inhibiting bacterial protein synthesis through a novel action.

In general, the invention provides a method for treating ocular infections such as endophthalmitis by systemically administering to a mammal, such as a human or animal, a pharmaceutical composition including a therapeutically effective amount of one or both compounds of formula I and II, or pharmaceutically acceptable salts thereof.

Surprisingly, the compound of formula I or II, or mixtures thereof, demonstrate excellent ocular penetration when administered systemically by passing through the blood-aqueous and blood-retinal barriers into the aqueous humor and vitreous. The amount of the compound of formula I or II, or mixtures thereof, in the aqueous humor and vitreous, after systemic administration, can reach concentrations sufficient to treat bacterial infections. The actual concentration of the compound of formula I or II, or mixtures thereof, in the aqueous humor and vitreous depends upon the systemic dosage. In some embodiments, an oral dose of a pharmaceutical composition containing 600 mg of the compound of formula I or II, or mixtures thereof, can result in aqueous humor and vitreous concentrations sufficient to treat bacterial infections. The exact concentration needed to treat bacterial infections depends both on the antimicrobial agent and the species of bacterium. In general, a sufficient aqueous humor and vitreous concentration of the compound of formula I or II, or mixtures thereof, for treating bacterial infections is about 4 μg/ml. Concentrations of the compound of formula I or II, or mixtures thereof below 4 μg/ml also may be effective in treating certain bacteria.

Without wishing to be bound to any particular theory, a hypothesis for the build-up of sufficient levels of the compound of formula I or II, or mixtures thereof, in the aqueous humor and vitreous includes penetration of the blood-aqueous and blood-retinal barriers and reduced or limited efflux pumping of the compound of formula I or II, or mixtures thereof, such as by p-glycoprotein pumps (PGP). Efflux pumping can reduce the concentration of compounds, such as antimicrobials, within the aqueous humor and vitreous by “pumping” or excluding the compound from those regions of the eye.

The compound of formula I or II, or mixtures thereof, may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylanine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydruiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

By the term “effective amount” of a compound as provided herein is meant a nontoxic but sufficient amount of the compound(s) to provide the desired effect As pointed out below, the exact amount required will vary from subject to subject, depending on the species, age, and general condition of the subject, the severity of the disease that is being treated, the particular compound(s) used, the mode of administration, and the like. Thus, it is not possible to specify an exact “effective amount.” However, an appropriate effective amount may be determined by one of ordinary skill in the art using only routine experimentation.

The therapeutically effective amount of the compound of formula I or II, or mixtures thereof, that is administered and the dosage regimen for treating a disease condition with the compound of formula I or II, or mixtures thereof, and/or compositions containing the compound of formula I or II, or mixtures thereof, depends on a variety of factors, including the age, weight, sex and medical condition of the subject, the severity of the disease, the route and frequency of administration, and the particular compound(s) employed, and thus may vary widely. The dosage of the compound of formula I or II, or mixtures thereof, as administered to a mammal can be between about 0.001 to about 100 mg/kg of body weight/day. In general, linezolid is a component of a pharmaceutical composition. Pharmaceutical compositions contain well known carriers and excipients in addition to the compound of formula I or II, or mixtures thereof. The pharmaceutical compositions may contain the compound of formula I or II, or mixtures thereof, in an amount in the range between about 1 to about 1000 mg, preferably in the range of between about 200 to about 800 mg. Typically, the pharmaceutical composition includes about 600 mg of the compound of formula I or II, or mixtures thereof. Generally the pharmaceutical composition includes between about 0.5% to about 90% by weight of the compound of formula I or II, or mixtures thereof. A total daily dose of about 1 to 1000 mg of the compound of formula I or II, or mixtures thereof, may be appropriate for an adult. The daily dose can be administered in one to four doses per day.

The desired dose may conveniently be presented in a single dose or as divided into multiple doses administered at appropriate intervals, for example, as two, three, four or more sub-doses per day. The sub-dose itself may be further divided, e.g., into a number of discrete loosely spaced administrations.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

Formulations for systemic administration may be in the form of aqueous solutions and suspensions, in addition to solid tablet and capsule formulations. The aqueous solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants are well and widely known in the pharmaceutical art. The compositions may, for example, be administered parenterally, e.g., intravascularly, intraperitoncally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions.

Pharmaceutical compositions of the compound of formula I or II, or mixtures thereof, either individually or in combination with other antimicrobial agents, may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For systemic administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient.

In addition to the compound of formula I or II, or mixtures thereof, the pharmaceutical composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutical acceptable materials. The components pharmaceutical composition can be encapsulated or tableted for convenient administration.

For oral administration, the pharmaceutical composition may be in the form of, for example, a tablet, capsule, suspension or liquid If desired, other active ingredients may be included in the composition.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

Alternatively, the compound of formula I or II, or mixtures thereof, may be in a powder form for constitution with a suitable vehicle, e.g., sterile, pyrogen-free water, before use.

For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

In some embodiments, the compound of formula I or II, or mixtures thereof, can be administrated by inhalation provided that the compounds pass into the blood stream. For example, pharmaceutical compositions containing the compound of formula I or II, or mixtures thereof, such as linezolid, can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

Additionally, the compound of formula I or II, or mixtures thereof, may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

The compound of formula I or II, or mixtures thereof, may also be delivered by controlled-release formulation as may be provided in a dispersion of active compound in hydroxypropyl-methyl cellulose, or other methods known to those skilled in the art.

The pharmaceutical compositions also may be part of a combination therapy. In a combination therapy, the compound of formula I or II or mixtures thereof, and other medicaments, such as other antimicrobial, anti-inflammatory, and pain relief agents, can be administered simultaneously or at separate intervals. When administered simultaneously the compound of formula I or II, or mixtures thereof, and other medicaments can be incorporated into a single pharmaceutical composition or into separate compositions, e.g., the compound of formula I or II, or mixtures thereof, in one composition and the other medicaments in another composition. Each of these compositions may be formulated with common excipients, diluents or carriers, and compressed into tablets, or formulated elixirs or solutions. The compounds can be formulated as sustained relief dosage forms and the like.

When separately administered, therapeutically effective amounts of the compound of formula I or II, or mixtures thereof, and the other medicaments are administered on a different schedule. One may be administered before the other as long as the time between the two administrations falls within a therapeutically effective interval. A therapeutically effective interval is a period of time beginning when one of either (a) the compound of formula I or II, or mixtures thereof, or (b) the other medicaments is administered to a mammal and ending at the limit of the beneficial effect in the treatment of ocular infection of the combination of (a) and (b).

The compounds of formula I and II may also be administered simultaneously or together.

Without further elaboration, it is believed that one skilled in the art can, using the preceding description, practice the present invention to its fullest extent. The foregoing detailed description is given for clearness of understanding only, and no unnecessary limitations should be understood therefrom, as modifications within the scope of the invention may become apparent to those skilled in the art.

1-19. (canceled)

20. A method of treating ocular infections comprising orally or intravenously administering to a mammal a therapeutically effective amount of the compound of formula I 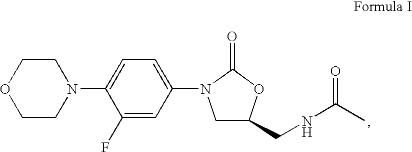
or a pharmaceutically acceptable salt thereof.

21. The method of claim 20 wherein the compound of formula I is administrated orally.

22. The method of claim 20 wherein the compound of formula I is administrated intravenously.

23. The method of claim 20 wherein the compound of formula I or its pharmaceutically acceptable salt thereof is a component of a pharmaceutical composition.

24. The method of claim 23 wherein the pharmaceutical composition comprising from about 0.5% to about 90% by weight of the compound of formula I or pharmaceutically acceptable salts thereof.

25. The method of claim 23 wherein the pharmaceutical composition comprising between about 1 mg and about 1000 mg of the compound of formula I or its pharmaceutically acceptable salt thereof.

26. The method of claim 23, wherein the pharmaceutical composition comprising between about 200 mg and about 800 mg of the compound of formula I or its pharmaceutically acceptable salts thereof.

27. The method of claim 23, wherein the pharmaceutical composition comprising about 600 mg of the compound of formula I or its pharmaceutically acceptable salt thereof.

28. The method of claim 23, wherein the pharmaceutical composition is administered as a tablet or capsule.

29. The method of claim 20 wherein the ocular infection is endophthalmitis.

30. The method of claim 20 wherein the therapeutically effective amount of the compound of formula I or its pharmaceutically acceptable salt thereof is administered to a mammal in an amount from about 0.1 to about 100 mg/kg of mammal body weight/day.

31. The method of claim 20 wherein the therapeutically effective amount of the compound of formula I or its pharmaceutically acceptable salt thereof is administered to a mammal in about 600 mg per day.

32. The method of claim 30 wherein the therapeutically effective amount is administered in more than one doses per day.

33. The method of claim 30 wherein the therapeutically effective amount is administered in one dose per day.
